(19) United States
(12) Patent Application Publication
(10) Pub. No.: US 2017/0079970 A1
(43) Pub. Date: Mar. 23, 2017

(54) TETRAHYDROQUINOLINE DERIVATIVES AND THEIR USE AS EPAC1 INHIBITORS FOR THE TREATMENT OF MYOCARDIAL INFARCTION INJURY

(71) Applicants: INSERM (INSTITUT NATIONAL DE LA SANTÉ ET DE LA RECHERCHE MÉDICALE, Paris (FR); UNIVERSITÉ PAUL SABATIER TOULOUSE III, Toulouse (FR)

(72) Inventors: Frank LEZOUALC'H, Paris (FR); Pierre SICARD, Paris (FR); Malik BISSERIER, Paris (FR)

(21) Appl. No.: 15/126,312

(22) PCT Filed: Mar. 20, 2015
(86) PCT No.: PCT/EP2015/055871

§ 371 Date: Sep. 15, 2016

(30) Foreign Application Priority Data
Mar. 21, 2014 (EP) 14305403.9

(51) Int. Cl. A61K 31/47; A61K 45/06

(52) U.S. Cl. CPC A61K 31/47; A61K 45/06

(57) ABSTRACT

The present invention relates to methods and pharmaceutical compositions for cardioprotection of subjects who experienced a myocardial infarction. In particular, the present invention relates to a method for providing cardioprotection in a subject who experienced a myocardial infarction comprising administering the subject with a therapeutically effective amount of at least one EPAC1 inhibitor.

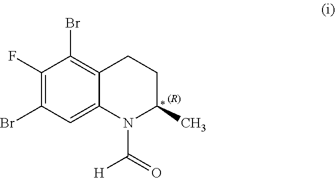

### FIELD OF THE INVENTION

The present invention relates to methods and pharmaceutical compositions for cardioprotection of subjects who experienced a myocardial infarction.

### BACKGROUND OF THE INVENTION

Myocardial infarction, commonly known as a heart attack, occurs when the blood supply to part of the heart is interrupted causing some heart cells to die. This is most commonly due to occlusion of a coronary artery following the rupture of a vulnerable atherosclerotic plaque. The resulting ischemia and oxygen shortage, if left untreated for a sufficient period of time, can cause damage and or death of heart muscle tissue. Accordingly, in clinical situations of myocardial infarction, the immediate goal is to restore blood flow to the patient as quickly as possible. If blood flow is restored within a suitable time period, tissue damage can be averted. However, a significant delay in restoring blood flow leads to a second condition known as ischemia-reperfusion injury that can develop gradually after an ischemic event and may cause irreversible damage to tissues. Clinical examples include cardiac contractile dysfunction, arrhythmias and irreversible myocyte damage (heart cell death) following myocardial infarction. Accordingly, several methods for the cardioprotection after myocardial infarction have been investigated. For example, current therapies aimed at improving contractile function often involve the use of inotropic agents (e.g., calcium, dopamine, epinephrine, ephedrine, phenylephrine, dobutamine). However inotropic drugs have been reportedly associated with increases in intracellular calcium concentration and heart rate, which may be potentially harmful, especially in hearts with impaired energy balance. Thus the limited successful for cardioprotection is limited by a relatively small number of therapeutic targets. The present invention fulfills this need by providing a new therapeutic target for the cardioprotection after myocardial infarction.

### SUMMARY OF THE INVENTION

The present invention relates to methods and pharmaceutical compositions for cardioprotection of subjects who experienced a myocardial infarction. In particular, the present invention is defined by the claims.

### DETAILED DESCRIPTION OF THE INVENTION

The present invention relates to a method for providing cardioprotection in a subject who experienced a myocardial infarction comprising administering the subject with a therapeutically effective amount of at least one EPAC1 inhibitor.

As used herein the term “cardioprotection” means protecting against or reducing damage to the myocardium after a myocardial infarction, after, during or prior to ischemic reperfusion. In particular, cardioprotection includes reducing infarct size, reducing ischemia-reperfusion injury, reducing hypoxia induced apoptosis/necrosis and preventing cardiomyocyte cell death. The method of the present invention is thus particularly suitable for the treatment of myocardial infarction injury in a subject in need thereof. As used herein, the term “treatment” refers to therapeutic treatment and prophylactic or preventative measures, wherein the object is to prevent or slow down (lessen) the targeted pathologic condition or disorder. A subject is successfully “treated” for myocardial infarction injury if, after receiving a therapeutic amount of the EPAC1 inhibitor, the subject shows observable and/or measurable reduction in or absence of one or more signs and symptoms of myocardial infarction injury, such as, e.g., reduced infarct size.

In some embodiments, the EPAC1 inhibitor is administered to a subject having one or more signs or symptoms of acute myocardial infarction injury. In some embodiments, the subject has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating.

In some embodiments, the EPAC1 inhibitor is administered simultaneously or sequentially (i.e. before or after) with a revascularization procedure performed on the subject. In some embodiments, the subject is administered with the EPAC1 inhibitor before, during, and after a revascularization procedure. In some embodiments, the subject is administered with the EPAC1 inhibitor as a bolus dose immediately prior to the revascularization procedure. In some embodiments, the subject is administered with the EPAC1 inhibitor continuously during and after the revascularization procedure. In some embodiments, the subject is administered with the EPAC1 inhibitor for a time period selected from the group consisting of at least 3 hours after a revascularization procedure; at least 5 hours after a revascularization procedure; at least 8 hours after a revascularization procedure; at least 12 hours after a revascularization procedure; at least 24 hours after a revascularization procedure. In some embodiments, the subject is administered with the EPAC1 inhibitor in a time period selected from the group consisting of starting at least 8 hours before a revascularization procedure; starting at least 4 hours before a revascularization procedure; starting at least 2 hours before a revascularization procedure; starting at least 1 hour before a revascularization procedure; starting at least 30 minutes before a revascularization procedure.

In some embodiments, the revascularization procedure is selected from the group consisting of percutaneous coronary intervention; balloon angioplasty; insertion of a bypass graft; insertion of a stent; directional coronary atherectomy; treatment with a one or more thrombolytic agent(s); and removal of an occlusion.

As used herein the term “EPAC” has its general meaning in the art and refers to the guanine exchange factor (GEF) Epac (Exchange Protein directly Activated by Cyclic AMP). There are two isoforms of Epac, Epac1 and Epac2, both consisting of a regulatory region binding directly cAMP and a catalytic region that promotes the exchange of GDP (Guanosine diphosphate) for GTP (Guanosine-5′-triphosphate) on the Ras-like small GTPases Rap1 and Rap2 isoforms). The two isoforms of Epac differ in that Epac1 has a single cyclic nucleotide-binding (CNB) domain, whereas Epac2 has two CNB domains, called CNB-A and CNB-B, which are located on both sides of the DEP domain (Dishevelled, Egl-10 and Pleckstrin domain). The additional N-terminal CNB domain in Epac2 has a low affinity for cAMP, and its deletion does not affect the regulation of Epac2 in response to agonists (J. de Rooij, H. Rehmann, M. van Triest, et al., Mechanism of regulation of the Epac family of cAMP-dependent RapGEFs, J. Biol. Chem. 275 (2000) 20829-20836).

As used herein the term “EPAC1 inhibitor” refers to any compound that is able to inhibit the activity or expression of EPAC1. In particular, a compound which leads to the inhibition of the Epac-induced Rap1, or Rap2 and Ras activation is a EPAC1 inhibitor. Even more a compound that inhibits Epac1 downstream effectors Rap1, or Rap2 and Ras following Epac activation by Epac1 agonists is an EPAC1 inhibitor.

In some embodiments, the EPAC1 inhibitor is a selective EPAC1 inhibitor. Epac1 selective inhibitors are compounds which exhibit an inhibitory effect on the Epac1 isoform. More particularly, they generally exhibit an inhibitory effect on Epac1 and moderate or no inhibitory effect on Epac2 isoform. By “selective Epac1 inhibitor” it may be understood the ability of the Epac1 inhibitors to affect the particular Epac1 isoform, in preference to the other iso form Epac2. The Epac1 selective inhibitors may have the ability to discriminate between these two isoforms, and so affect essentially the Epac1 isoform. In some embodiments, they may exhibit a ratio of inhibition of Epac1 versus Epac2 of at least 10 folds.

EPAC1 inhibitors are well known in the art (Courilleau D, Bouyssou P, Fischmeister R, Lezoualc'h F, Blondeau J P. The (R)-enantiomer of CE3F4 is a preferential inhibitor of human exchange protein directly activated by cyclic AMP isoform 1 (Epac1). Biochem Biophys Res Commun. 2013 Oct. 25; 440(3):443-8; Courilleau D, Bisserier M, Jullian J C, Lucas A, Bouyssou P, Fischmeister R, Blondeau J P, Lezoualc'h F. Identification of a tetrahydroquinoline analog as a pharmacological inhibitor of the cAMP-binding protein Epac. J Biol Chem. 2012 Dec. 28; 287(53):44192-202; Brown L M, Rogers K E, McCammon J A, Insel P A. Identification and Validation of Modulators of Epac Activity: Structure-function Implications for Epac Activation and Inhibition. J Biol Chem. 2014 Feb. 4.).

In some embodiments, the EPAC1 inhibitor is a tetrahydroquinoline derivative. In some embodiments, the EPAC1 inhibitor is a tetrahydroquinoline derivative having formula (I):

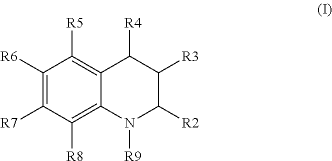

wherein:
R9 is H or

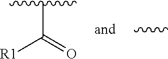

is the attachment to the nitrogen atom of the tetrahydroquinoline;
R1, R2, R3, R4 and R8 are independently chosen from the group consisting of H, (C₁-C₁₀)alkyl, (C₃-C₁₀)cycloalkyl, (C₆-C₁₀)aryl, (C₁-C₆)alkylene-(C₆-C₁₀)aryl and (C₃-C₁₀)heteroaryl; said aryl and heteroaryl groups being possibly substituted by at least one substituent chosen from OH, NH₂, NO₂, (C₁-C₆)alkyl and halogen;
R5 is an halogen atom;
R6 and R7 are independently chosen from the group consisting of H and halogen atoms;
or its pharmaceutically acceptable salts, hydrates or hydrated salts or its polymorphic crystalline structures, racemates, diastereomers or enantiomers,

The term “(C₁-C₁₀)alkyl” means a saturated or unsaturated aliphatic hydrocarbon group which may be straight or branched having 1 to 10 carbon atoms in the chain. Preferred alkyl groups have 1 to 4 carbon atoms in the chain, preferred alkyl groups are in particular methyl or ethyl groups. “Branched” means that one or lower alkyl groups such as methyl, ethyl or propyl are attached to a linear alkyl chain.

The term “(C₁-C₆)alkylene-” means a saturated or unsaturated aliphatic hydrocarbon divalent radical which may be straight or branched having 1 to 6 carbon atoms in the chain. For example, a preferred (C₁-C₆)alkylene-(C₆-C₁₀)aryl is a benzyl group.

By “(C₃-C₁₀)cycloalkyl” is meant a cyclic, saturated hydrocarbon group having 3 to 10 carbon atoms, in particular cyclopropyl or cyclohexyl groups.

The term “(C₆-C₁₀)aryl” refers to an aromatic monocyclic, bicyclic, or tricyclic hydrocarbon ring system wherein any ring atom capable of substitution may be substituted by a substituent. Examples of aryl moieties include, but are not limited to, phenyl.

The term “(C₃-C₁₀)heteroaryl” refers to an aromatic monocyclic, bicyclic, or tricyclic hydrocarbon ring system, wherein any ring atom capable of substitution may be substituted by a substituent and wherein one or more carbon atom(s) are replaced by one or more heteroatom(s) such as nitrogen atom(s), oxygen atom(s) and sulphide atom(s); for example 1 or 2 nitrogen atom(s), 1 or 2 oxygen atom(s), 1 or 2 sulphide atom(s) or a combination of different heteroatoms such as 1 nitrogen atom and 1 oxygen atom. Preferred heteroaryl groups are pyridyl, pyrimydyl and oxazyl groups.

The term “halogen” refers to the atoms of the group 17 of the periodic table and includes in particular fluorine, chlorine, bromine, and iodine atoms, more preferably fluorine, chlorine and bromine atoms.

By “tetrahydroquinoline” it is understood the following group:

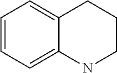

The compounds of formula (I) herein described may have asymmetric centers. Compounds of formula (I) containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated. In one embodiment, the carbon atom referred to with (*) in the formula (I) with R2 to R9 as defined above may be (R) or (S):

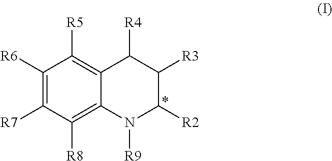

In one embodiment it is (R). In some embodiments, the enantiomeric form (R) of the compound of formula (I) is preferred and more particularly the following enantiomeric form:

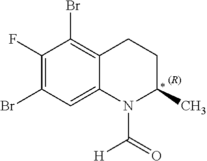

In one embodiment, the (R)-enantiomeric form of the compound of formula (I) is a selective inhibitor of Epac1. Said (R)-enantiomeric form may inhibit the GEF activity of Epac1 with 10-times more efficiency than the (S)-enantiomeric form.

The term “pharmaceutically acceptable salt” refers to salts which retain the biological effectiveness and properties of the compounds of formula (I) and which are not biologically or otherwise undesirable. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

In some embodiments, the compounds of formula (I) have the following formula:

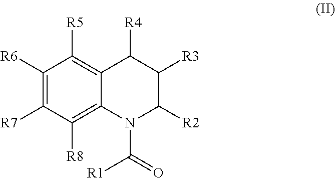

that is in formula (I), R9 is

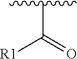

In some embodiments, the compounds of formula (I) have the following formula (II):

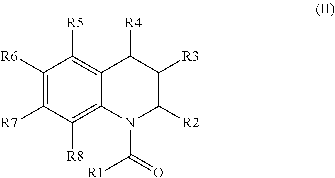

Wherein
R1, R2, R3, R4 and R8 are independently chosen from the group consisting of H, (C₁-C₁₀)alkyl, (C₃-C₁₀)cycloalkyl, (C₆-C₁₀)aryl, (C₁-C₆)alkylene-(C₆-C₁₀)aryl and (C₃-C₁₀)heteroaryl; said aryl and heteroaryl groups being possibly substituted by at least one substituent chosen from OH, NH₂, NO₂, (C₁-C₆)alkyl and halogen;
R5 is an halogen atom;
R6 and R7 are independently chosen from the group consisting of H and halogen atoms;
or its pharmaceutically acceptable salts, hydrates or hydrated salts or its polymorphic crystalline structures, racemates, diastereomers or enantiomers,

In some embodiments, in formula (II) as defined above R1 is H. In some embodiments, R2 is H or a (C₁-C₁₀)alkyl. In some embodiments, R2 is a (C₁-C₁₀)alkyl. Preferably, R2 is a (C₁-C₄)alkyl. More preferably, R2 is a methyl group. In another embodiment, R2 is H. Preferably, R2 is H or a methyl group. In some embodiments, R3 is H. In another embodiment, R4 is H. In another embodiment, R8 is H. In one embodiment, R3, R4 and R8 are H. In some embodiments, the (C₃-C₁₀)heteroaryl group is chosen from the group consisting of pyridyl, pyrimydyl and oxazyl groups. In another embodiment, the (C₆-C₁₀)aryl group is a phenyl group. In another embodiment, the (C₁-C₆)alkylene-(C₆-C₁₀)aryl is a benzyl group. In some embodiments, R5 is chosen from the group consisting of F, Cl, Br and I. Preferably, R5 is Br. In some embodiments, R6 is chosen from the group consisting of H, F, Cl, Br and I. In some embodiments, R6 is chosen from the group consisting of F, Cl, Br and I. In another embodiment, R6 is F. In another embodiment R6 is H. Preferably, R6 is H or F. In some embodiments, R7 is chosen from the group consisting of H, F, Cl, Br and I. In some embodiments, R7 is chosen from the group consisting of F, Cl, Br and I. In another embodiment, R7 is Br. In another embodiment, R7 is H. Preferably, R7 is H or Br. In a preferred embodiment, R1 is H and R5 is Br. In another preferred embodiment, at least two of R5, R6 and R7 are halogen.

Some specific compounds have the following formulae:

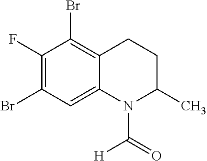 [SMILES: CC1CCc2c(cc(Br)c(F)c2Br)N1C=O]

named herein CE3F4,

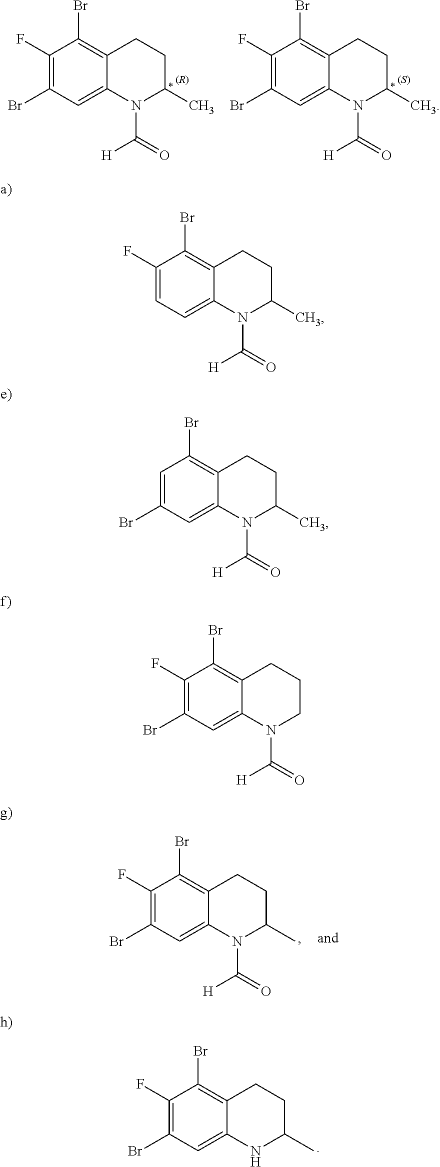

More particularly, some specific compounds have the following formulae:

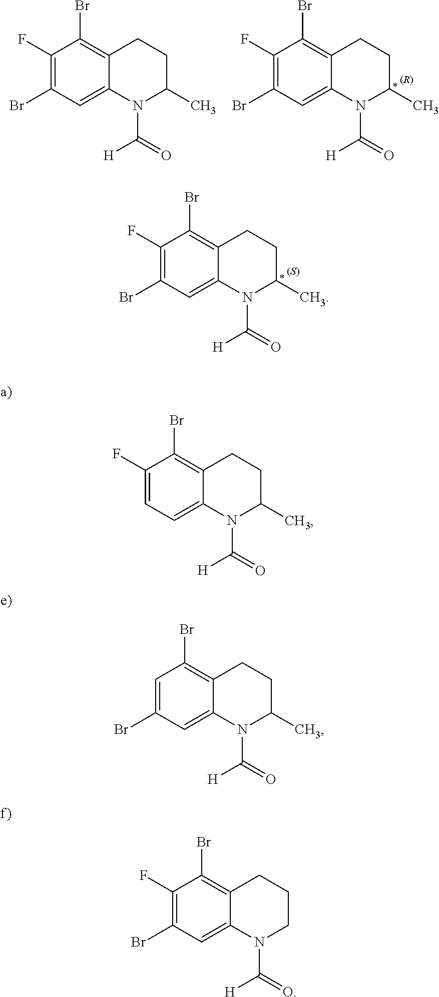

In one embodiment, the compound of formula (I) is:

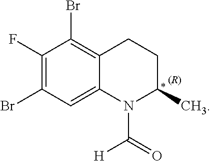

The compounds of formula (I) can be synthesized according to previously published methods in P. Bouyssou et al., J. Heterocyclic Chem., 29, 895, 1992. Methods of preparation of the compounds of formula (I) are well-known.

In some embodiments, the EPAC1 inhibitor is an inhibitor of EPAC1 expression. An “inhibitor of expression” refers to a natural or synthetic compound that has a biological effect to inhibit the expression of a gene. Therefore, an “inhibitor of EPAC1 expression” denotes a natural or synthetic compound that has a biological effect to inhibit the expression of EPAC1 gene. In some embodiments, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of EPAC1 mRNA by binding thereto and thus preventing protein translation or increasing mRNA degradation, thus decreasing the level of EPAC1, and thus activity, in a cell. For example, antisense oligonucleotides of at least about 15 bases and complementary to unique regions of the mRNA transcript sequence encoding EPAC1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EPAC1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991). Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In some embodiments, the EPAC1 inhibitor is administered in combination with an additional active agent. In some embodiments, the additional active agent is a cardiovascular agent selected from the group consisting of hyaluronidase, a corticosteroid, recombinant superoxide dismutase, prostacyclin, fluosol, magnesium, poloxamer 188, trimetazidine, eniporidine, cariporidine, a nitrate, anti-P selectin, an anti-CD18 antibody, adenosine, and glucose-insulin-potassium. In some embodiments, the cardiovascular agent is selected from the group consisting of an anti-arrhthymia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a throboxane receptor antagonist, a radical scavenger, an anti-platelet drug, a β-adrenaline receptor blocking drug, oreceptor blocking drug, a sympathetic nerve inhibitor, a digitalis formulation, an inotrope, and an antihyperlipidemic drug. In some embodiments, the active agent is an inotrope. Positive inotropic agents increase myocardial contractility, and are used to support cardiac function in conditions such as decompensated congestive heart failure, cardiogenic shock, septic shock, myocardial infarction, cardiomyopathy, etc. Examples of positive inotropic agents include, but are not limited to, Berberine, Bipyridine derivatives, Inamrinone, Milrinone, Calcium, Calcium sensitizers, Levosimendan, Cardiac glycosides, Digoxin, Catecholamines, Dopamine, Dobutamine, Dopexamine, Epinephrine (adrenaline), Isoprenaline (isoproterenol), Norepinephrine (noradrenaline), Eicosanoids, Prostaglandins, Phosphodiesterase inhibitors, Enoximone, Milrinone, Theophylline, and Glucagon. Negative inotropic agents decrease myocardial contractility, and are used to decrease cardiac workload in conditions such as angina. While negative inotropism may precipitate or exacerbate heart failure, certain beta blockers (e.g. carvedilol, bisoprolol and metoprolol) have been shown to reduce morbidity and mortality in congestive heart failure. Examples of negative inotropic agents include, but are not limited to, Beta blockers, Calcium channel blockers, Diltiazem, Verapamil, Clevidipine, Quinidine, Procainamide, disopyramide, and Flecainide. In some embodiments, the cardiovascular agent is cyclosporine. As used herein, the term “cyclosporine” refers to cyclosporine A, cyclosporine G, and functional derivatives or analogues thereof, e.g., NIM81 1. Cyclosporine A refers to the natural Tolypocladium inflation cyclic non-ribosomal peptide. Cyclosporine G differs from cyclosporine A in the amino acid 2 position, where an L-norvaline replaces the a-aniinobutyric acid. (See generally, Wenger, R. M. 1986. Synthesis of Ciclosporin and analogues: structural and conformational requirements for immunosuppressive activity. Progress in Allergy, 38:46-64).

As used herein, the term “effective amount” refers to a quantity sufficient to achieve cardioprotection. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. In some embodiments, an effective amount of the EPAC1 inhibitors for achieving a therapeutic or prophylactic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In some embodiments, a single dosage of peptide ranges from 0.1-10,000 micrograms per kg body weight. In some embodiments, aromatic-cationic peptide concentrations in a carrier range from 0.2 to 2000 micrograms per delivered milliliter.

Typically, The EPAC1 inhibitor is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the EPAC1 inhibitor in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1: Epac1−/− mice are more resistant to acute myocardial infarction after permanent ischemia or ischemia/reperfusion injury

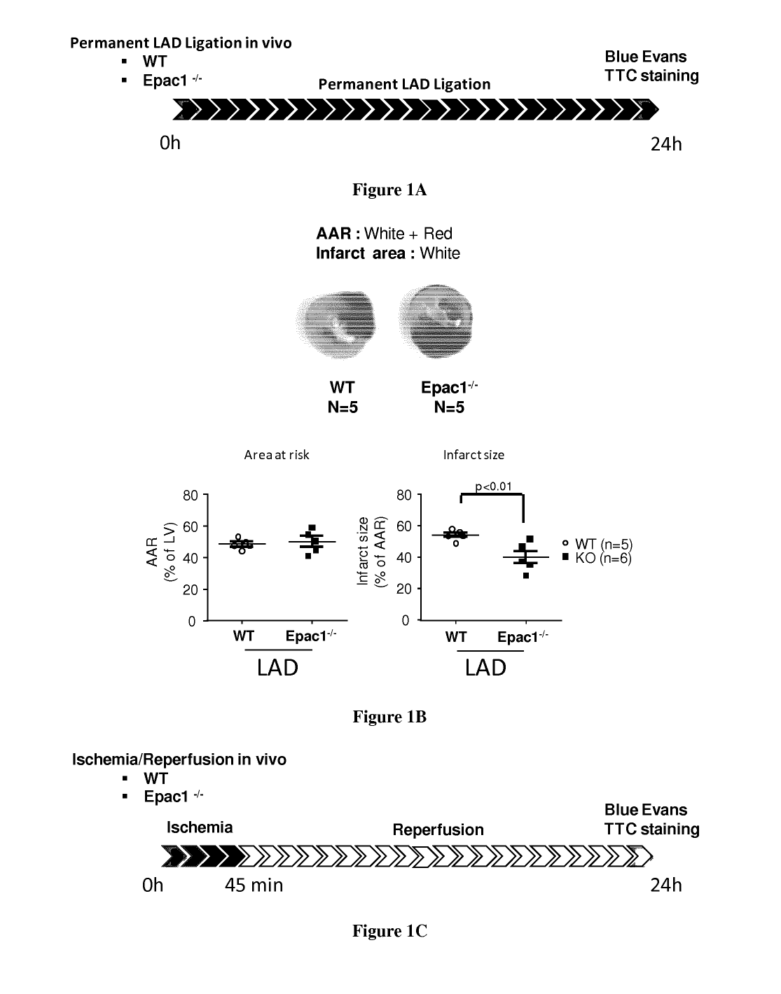

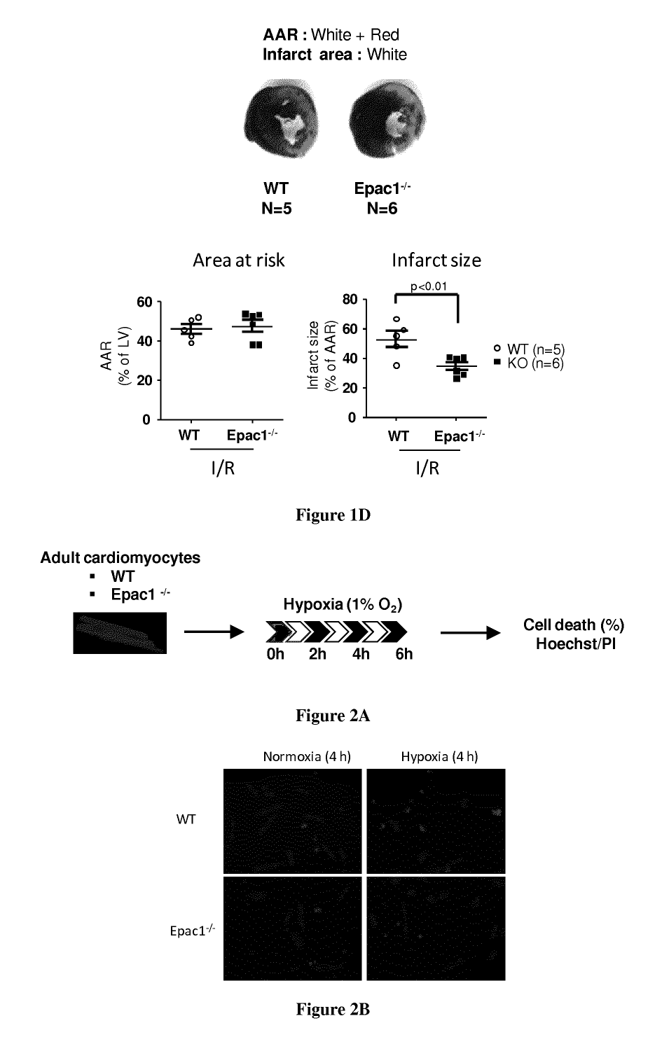

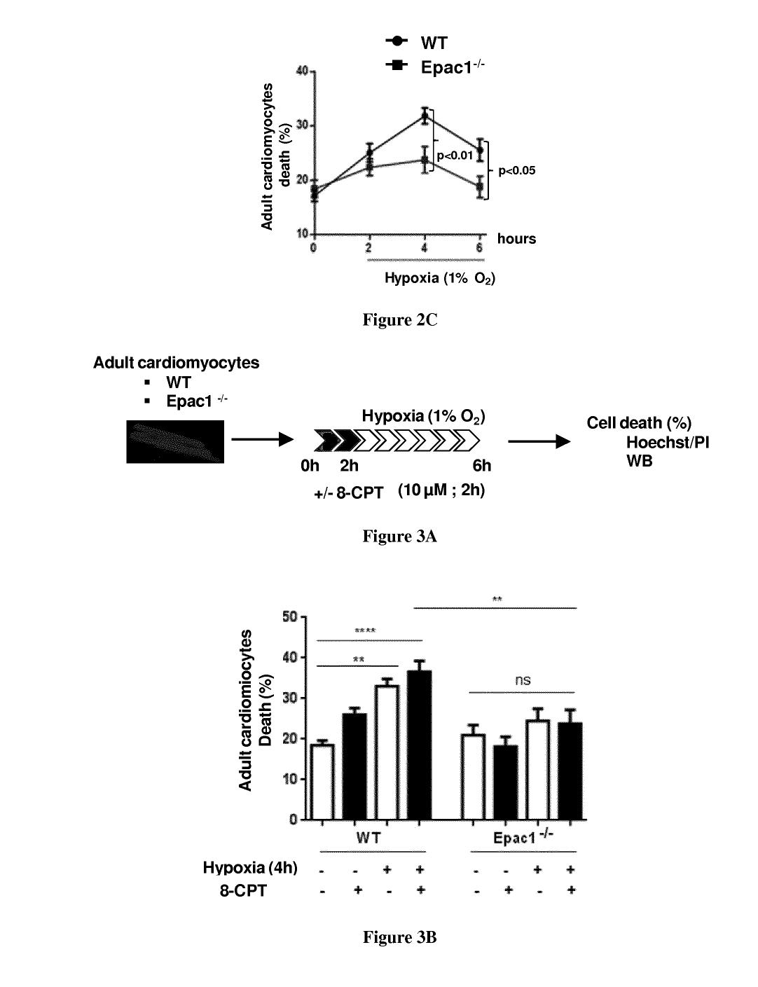

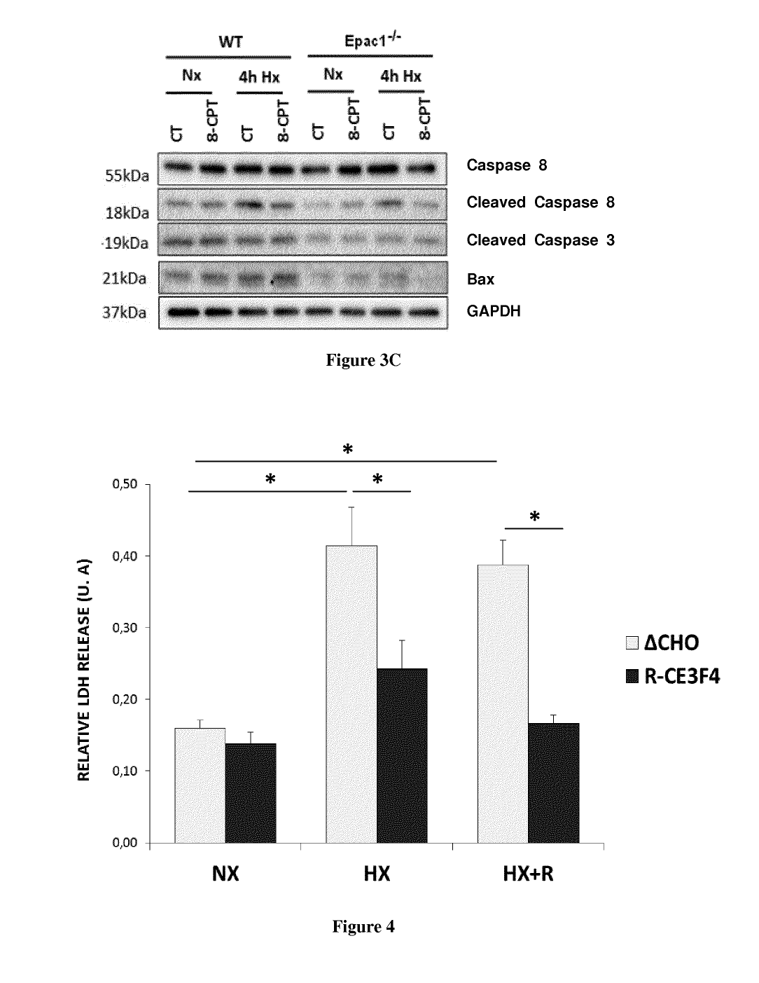

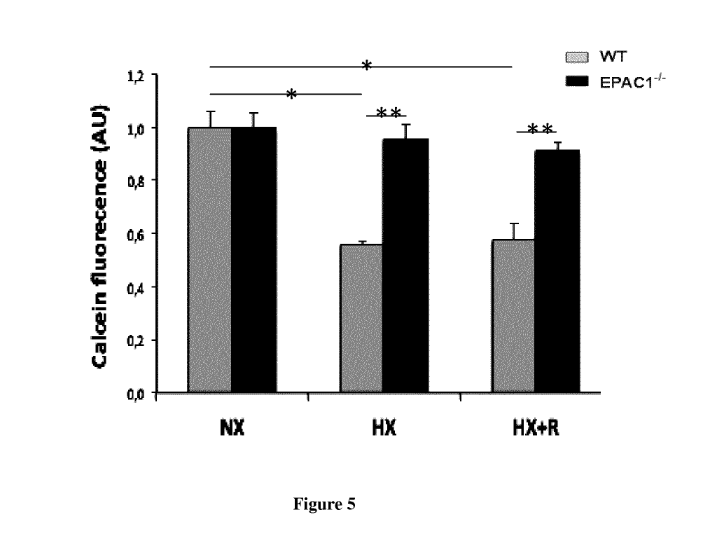

A. Myocardial infarction was induced by permanent ligation of the left anterior descending coronary artery in the WT and Epac1^{-/-} mice. Infarct size was examined by 2,3,5-triphenyltetrazolium (TTC) staining One day after LAD ligation, the hearts were removed and perfused with saline solution before staining with 1% Evans Blue. The hearts were then sliced and incubated in 1% TTC for 15 min at 37° C.

B. Representative pictures showing myocardial infarct size in heart sections defined by dual staining with (TTC) and Evans blue. Bar graphs show the ratio of the area at risk (AAR) to myocardium, and the ratio of infarct size to AAR at 24 hours post-LAD ligation in WT and Epac1^{-/-} animals. Results are expressed as means±S.E.M. Note that the area at risk was not different among the groups. Epac1^{-/-} mice showed a reduction in infarct size compared with WT control mice.

C. In vivo regional ischemia was induced in the WT and Epac1 mice where the LAD was occluded for 45 min and followed by 24 h of reperfusion. At the end of the reperfusion, myocardial infarct size was measured as indicated above.

D. Representative pictures show myocardial infarct size in heart sections defined by dual staining with (TTC) and Evans blue after ischemia/reperfusion injury. The infarct size is significantly smaller in Epac1^{-/-} mice compared to the WT animals despite the same AAR.

FIG. 2. Epac1 deletion as a protective effect on cardiomyocytes death during hypoxia

A. Adult ventricular cardiomyocytes were isolated from WT and Epac1^{-/-} mice. After plating, cells were then exposed in either normoxic or hypoxic condition for 2 h, 4 h or 6 h (1% O₂). Cell death was determined by Hoechst/Propidium Iodide staining

B. Representative images of hypoxia induced cell death in WT and Epac1−/− cardiomyocytes.

C. Hypoxia increased cardiomoycytes death in WT cardiomyocytes but not in Epac1^{-/-} cardiomyocytes. The ratio of dead cells to total cells in WT and Epac1^{-/-} cardiomyocytes was calculated at 2 h, 4 h or 6 h of hypoxia. Results are expressed as means±S.E.M.

FIG. 3: Hypoxia and the Epac agonist, 8-CPT increase cardiomyocytes death and the content of apoptotic proteins in an Epac1 dependent manner

A. Adult ventricular cardiomyocytes were isolated from WT and Epac1 mice and cultured. Cell were treated with or without 8-CPT (10 μM) for 2 h and then exposed in normoxic or hypoxic condition for 4 h (1% O₂). Cell death was determined by Hoechst/Propidium Iodide staining and pro-apototic proteins expressions were concomitantly measured by western blot.

B. The 8-CPT treatment associated with 4 h of hypoxia increased dramatically the cardiomoycytes death in WT but not in the Epac1^{-/-} cardiomyocytes.

C. Hypoxia increases caspase 8 and 3 cleavage and Bax expression in WT cardiomyocytes but not in Epac1^{-/-} cardiomyocytes. Representative immunoblots of native caspase-8, cleaved caspase-8, cleaved caspase 3 and Bax after 4 h of normoxia or hypoxia in the presence or absence of 8-CPT

FIG. 4. A pharmacological inhibitor of Epac1, R-CE3F4 prevents hypoxia and hypoxia-reoxygenation induced cardiomyocyte death. Isolated mouse adult cardiomyocytes, pretreated or not with either 10 μM R-CE3F4 or its analogue ΔCHO (10 μM) were exposed to hypoxia (HX) for 4 h followed by a 2 h of reoxygenation period (HX+R). Cell death was determined by LDH release. Results are expressed as means±S.E.M. The bar graph represents the average of 5 independent experiments performed in triplicate. Statistical significance was determined by 2-way ANOVA. * P<0.05 compared with indicated values.

FIG. 5. Deletion of Epac1 prevents hypoxia-induced mitochondrial permeability transition pore (mPTP) opening. Isolated cardiomyocytes of WT and Epac1^{-/-} mice were loaded with calcein-AM and CoCl₂. mPTP opening was monitored by mitochondrial calcein release in normoxia (NX), hypoxia (HX) or hypoxia-reoxygenation conditions (HX+R). Results are expressed as means±S.E.M. The bar graph represents the average of 9 independent experiments. Statistical significance was determined by 2-way ANOVA. *P<0.05, ** P<0.01 compared with indicated values.

### EXAMPLE

### Exchange Protein Directly Activated by cAMP 1 (Epac1) Knock-Down Limits Cardiomyocytes Death During Myocardial Infarction and Ischemia/Reperfusion

Introduction

Ischemia/reperfusion is accompanied and influenced by perturbations of the beta-adrenergic receptor pathway and acts through diverse signaling cascades to modulate cardiac function and remodelling. The Epac1-Rap signaling pathway is a potent regulator of Ca²⁺ cycling, cardiac hypertrophy and fibrosis. However, the role of Epac1 in cardiomyocyte death remains underexplored. Here we investigated whether Epac1 knock-out (Epac1^{-/-}) mice were protected against myocardial infarction- and Ischemia/Reperfusion induced cell death.

Methods and Results

Myocardial infarction was induced in wild-type (WT) versus Epac1^{-/-} littermates by left anterior descending coronary artery (LAD) ligation for 24 h. Ischemia/Reperfusion was induced by the left anterior descending coronary artery occlusion for 45 min and followed by 24 h of reperfusion. The area at risk (AAR) and infarct size were evaluated by Evans blue and TTC staining, respectively. In both models, we found that the infarct size was significantly decreased in Epac1^{-/-} mice compared to the WT animals despite the same AAR. Concomitantly, adult cardiomyocytes isolated from the Epac1^{-/-} and WT mice were exposed to hypoxia for 2 h, 4 h or 6 h and cell death was determined by Hoechst/Propidium Iodide staining Our data show that hypoxia induced apoptosis/necrosis was prevented in Epac1 deleted cardiomyocytes (32% vs 24%). In addition, necroptosis activity pathway (Caspase 8; Caspase 3; Bax) as measured by Western blot was also reduced in the Epac1^{-/-} mice. Then, we investigated the effect of the Epac1 pharmacological inhibitor, R-CE3F4 and its inactive analogue (ΔCHO) on hypoxia- or hypoxia-reperfusion-induced cell death. Isolated mouse adult cardiomyocytes pretreated with either R-CE3F4 or (ΔCHO) were exposed to hypoxia (HX) for 4 h followed by a 2 h of reoxygenation period (HX+R). Cell death was determined by LDH release. Our data showed that HX− and HX+R-induced cardiomyocyte death were significantly prevented in cardiomyocytes pretreated with R-CE3F4. Finally, we found that deletion of Epac1 prevented HX− or HX+R induced mitochondrial permeability transition pore opening (mPTP).

Conclusion

Genetic deletion or pharmacological inhibition of Epac1 confers resistance to ischemic injury and ischemia-reperfusion, at least in part, via limiting cell death pathway in cardiomyocytes. Further studies are necessary to unveil the mechanism by which Epac1 promotes cardiomyocytes cell death. Epac1 pharmacological inhibitors seem to be good candidates to limit myocardial infarction injury.

### REFERENCES

Throughout this application, various references describe the state of the art to which this invention pertains. The disclosures of these references are hereby incorporated by reference into the present disclosure.

1. A method for providing cardioprotection in a subject who experienced a myocardial infarction comprising administering to the subject a therapeutically effective amount of at least one EPAC1 inhibitor.

2. (canceled)

3. The method of claim 1 wherein the EPAC1 inhibitor is administered simultaneously or sequentially with a revascularization procedure performed on the subject.

4. The method of claim 3 wherein the EPAC1 inhibitor is administered to the subject before, during, and after a revascularization procedure.

5. The method of claim 3 wherein the EPAC1 inhibitor is administered to the subject as a bolus dose immediately prior to the revascularization procedure.

6. The method of claim 3 wherein the EPAC1 inhibitor is administered to the subject for a time period selected from the group consisting of at least 3 hours after a revascularization procedure; at least 5 hours after a revascularization procedure; at least 8 hours after a revascularization procedure; at least 12 hours after a revascularization procedure; and at least 24 hours after a revascularization procedure.

7. The method of claim 3 wherein the revascularization procedure is selected from the group consisting of percutaneous coronary intervention; balloon angioplasty; insertion of a bypass graft; insertion of a stent; directional coronary atherectomy; treatment with a one or more thrombolytic agent(s); and removal of an occlusion.

8. The method of claim 1 wherein the EPAC1 inhibitor is a selective EPAC1 inhibitor.

9. The method of claim 1 wherein the EPAC1 inhibitor is a tetrahydroquinoline derivative having formula (I): 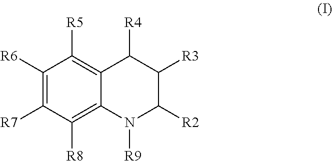
wherein:
R9 is H or 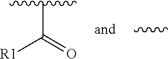
is the attachment to the nitrogen atom of the tetrahydroquinoline;
R1, R2, R3, R4 and R8 are independently selected from the group consisting of H, (C1-C10)alkyl, (C3-C10)cycloalkyl, (C6-C10)aryl, (C1-C6)alkylene-(C6-C10)aryl and (C3-C10)heteroaryl; said aryl and heteroaryl groups being optionally substituted by at least one substituent selected from the group consisting of OH, NH₂, NO₂, (C1-C6)alkyl and halogen;
R5 is an halogen atom;
R6 and R7 are independently H or halogen;
or a pharmaceutically acceptable salt, hydrate or hydrated salt thereof, or a polymorphic crystalline structure, racemate, diastereomer or enantiomer thereof.

10. The method of claim 9 wherein the EPAC1 inhibitor is an enantiomeric form (R) of the compound of formula (I).

11. The method of claim 9 wherein the EPAC1 inhibitor is

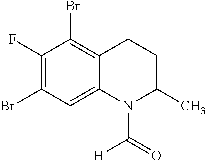 [SMILES: CC1CCc2c(cc(Br)c(F)c2Br)N1C=O]

12. The method of claim 9 wherein the EPAC 1 inhibitor is

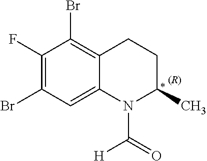

13. The method of claim 1 wherein the EPAC1 inhibitor is administered to the subject in combination with an additional active agent selected from the group consisting of an anti-arrhthymia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a throboxane receptor antagonist, a radical scavenger, an anti-platelet drug, a β-adrenaline receptor blocking drug, a sympathetic nerve inhibitor, a digitalis formulation, an inotrope, and an antihyperlipidemic drug.
